(19) United States
(12) Patent Application Publication
(10) Pub. No.: US 2019/0209712 A1
(43) Pub. Date: Jul. 11, 2019

(54) HUMANIZED MURINE MODEL OF CHRONIC HEPATITIS B CONSTRUCTED USING STEM CELLS AND METHOD OF USING SAME

(71) Applicant: Zhejiang University, Hangzhou (CN)

(72) Inventors: Jun Li, Hangzhou (CN); Suwan Sun, Hangzhou (CN); Jiang Li, Hangzhou (CN); Jing Jiang, Hangzhou (CN); Ningshao Xia, Hangzhou (CN); Tong Cheng, Hangzhou (CN); Lunzhi Yuan, Hangzhou (CN)

(21) Appl. No.: 16/355,723

(22) Filed: Mar. 16, 2019

Related U.S. Application Data
(63) Continuation-in-part of application No. PCT/CN2017/081751, filed on Apr. 24, 2017.

(30) Foreign Application Priority Data
Mar. 7, 2017 (CN) 201710130633.8

(51) Int. Cl. A61K 49/00; A61K 35/28; A61K 35/76; A01K 67/027

(52) U.S. Cl. CPC A61K 49/0008; A61K 35/28; A01K 2267/0337; A01K 67/0278; A01K 2227/105; A61K 35/76

(57) ABSTRACT

A humanized murine model of chronic hepatitis B constructed using human stem cells, obtained by obtaining the human stem cells; transplanting the human stem cells into a murine with liver damage to achieve presence of 1×10⁴⁻⁸ of the human stem cells in the murine in vivo; etc. The humanized murine model may be used for the study of drug resistance to the antiviral drugs, the study of the pathogenesis of chronic hepatitis B, etc. In addition to the study of the mechanism of hepatitis B viral infection, the humanized murine model may also be used for research in treatment.

### CROSS REFERENCE TO RELATED APPLICATIONS

This application is a continuation-in-part application of PCT Application No. PCT/CN2017/081751 filed on Apr. 24, 2017, which claims priority to Chinese Patent Application No. 201710130633.8 filed on Mar. 7, 2017, the entire contents of which are incorporated herein by reference.

### FIELD OF TECHNOLOGY

The present invention relates to the fields of clinical medicine, experimental medicine, regenerative medicine and virology, and specifically relates to a humanized murine model of chronic hepatitis B constructed based on stem cell technology.

### BACKGROUND

Hepatitis B virus (HBV) has a wide prevalence. According to the statistics, there are 100 million people infected with HBV in China. Since chronic hepatitis B cannot be completely cured, it imposes a heavy burden on the society, families and patients. At the same time, since hepatitis B virus can only cause diseases in high-level primates, the use of apes is often required in the animal testing models. Experiment with apes has a high cost, complicated operation, and long test cycle. Meanwhile, duck model of hepatitis B, which is more commonly used at the present time, is based on a poultry vehicle and differs greatly from the human species, hence having greater limitations. Since chronic hepatitis B is difficult to cure, according to the World Health Organization report in Year 2015, about 30% of the hepatitis B patients will develop liver cirrhosis and liver tumors, which pose a major threat to the health of the patients. Meanwhile, the existing commonly used animal models of liver cirrhosis are based on surgery (such as common bile duct ligation) and chemical drugs (carbon tetrachloride, dimethylnitrosamine, etc.). This type of model of liver cirrhosis is not caused by hepatitis B virus. Thus, the mechanism and outcome of the disease are very different from the liver cirrhosis caused by hepatitis B, and it is not suitable for the study of the mechanism and treatment of the liver fibrosis and liver cirrhosis caused by hepatitis B. Among the liver tumors patients in China, primary liver tumors caused by hepatitis B account for the vast majority of liver tumors cases. Primary liver tumors is one of the most common malignant tumors in China with a high mortality rate and is difficult to diagnose at the early stage. Other models of liver tumors constructed using small animals are mostly based on chemical drugs. Such models are very different from the model of liver tumors based on HBV infection. Particularly, there is a significant difference in the pathogenesis.

Therefore, it is of great scientific significance and application value to establish small mammal models which are susceptible to hepatitis B viral infection and diseases. However, since small mammals cannot be infected by hepatitis B virus, it is difficult to establish small mammal models for research, which has severely hindered the researches on the progression mechanism, treatment, and outcome of liver diseases, and has greatly restricted the optimization of the treatment options. The establishment of a humanized murine model of chronic hepatitis B will provide a good research vehicle for the study of the mechanism of viral hepatitis, and the treatment and disease outcome thereof. Bone marrow mesenchymal stem cells have potential ability to differentiate and may be differentiated into mature hepatocytes. In our previous study, it was found that human bone marrow mesenchymal stem cells (hBMSCs) and murine liver with liver damage may form chimera with a chimeric rate of as high as 50-95%. This laid the foundation for establishing a new type of humanized murine model. This murine model provides a safeguard for elucidating the biological characteristics of chronic hepatotropic B, the pathogenesis, the research and development, the screening of new drugs, etc. This murine model provides a good research vehicle for the study of hepatitis, and the treatment and disease outcome thereof.

### SUMMARY OF THE INVENTION

In view of the deficiency of the existing models of primary liver tumors, the present invention provides a humanized murine model of chronic hepatitis B. The present invention is realized by the following technical solutions:

The present invention discloses a humanized murine model of chronic hepatitis B constructed using human stem cells, wherein the humanized murine model is obtained by the following steps:
(a) obtaining the human stem cells;
(b) transplanting the human stem cells into a murine with liver damage to achieve presence of 1×10⁴⁻⁸ of the human stem cells in the murine in vivo;
(c) forming chimera of human-derived hepatocytes, wherein the human stem cells transplanted in the step (b) differentiate into the human-derived hepatocytes in a liver of the murine; and forming a humanized immune system, wherein the human stem cells transplanted in the step
(b) differentiate and form the human-derived immune system in the murine in vivo;
(d) infecting the murine with hepatitis B virus (HBV);
(e) confirming a HBV infection and a liver disease in the murine.

In a preferred embodiment, the human stem cells originated from stem cells isolated and cultured from a healthy human, or a commercialized cell line.

In a preferred embodiment, the liver damage comprises chemical liver damage caused by a chemical drug, physical liver damage caused by surgery or both; wherein the murine is a normal mouse, an immunodeficient mouse, a normal rat, or an immunodeficient rat.

In a preferred embodiment, in the step (c), the human stem cells which differentiate into the human-derived hepatocytes express a human hapatocytic marker selected from the group consisting of HSA, HLA, ALB and NTCP.

In a preferred embodiment, in the step (c), the human-derived immune system formed by the human by the human stem cells is capable of detecting an immune cell expressing a human lymphocytic marker selected from the group consisting of CD45⁺, CD4⁺, CD8⁺, CD3⁺, CD19⁺, CD20⁺, CD68⁺and NKp46⁺.

In a preferred embodiment, in the step (e), the HBV infection is confirmed by analyzing HBV DNA, HBsAg, HBeAg, HBcrAg HBsAb, HBeAb and HBcAb in serology, HBV cccDNA in hepatocytes and detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

In a preferred embodiment, the hepatitis is confirmed when a symptom comprising punctate necrosis is present and normal central-portal relationships is lost in the liver of the murine; the liver fibrosis is confirmed when inflammation and fibrosis are present in the liver of the murine; the liver cirrhosis is confirmed when a characteristic of the liver cirrhosis comprising change in pseudo lobule is found; the liver tumors is confirmed when a lesion of primary liver tumors observable by histology and imaging is present in the murine in vivo.

The present invention further discloses a method of researching and developing an antiviral drug, an anti-fibrosis drug, or an anti-tumor drug, comprising:
screening and testing the antiviral drug, anti-fibrosis drug, or anti-tumor drug using the humanized murine model according to the present invention.

The present invention further discloses a method of studying drug resistance to an antiviral drug, comprising:
testing the drug resistance to the antiviral drug using the humanized murine model according to the present invention.

The present invention further discloses a method of studying pathogenesis of chronic hepatitis B, comprising:
analyzing the pathogenesis of the chronic hepatitis B using the humanized murine model according to the present invention.

Comparing to the existing models, the advantageous effects of the present invention are as follows:

The human stem cells (such as human mesenchymal stem cells, human embryonic stem cells, and induced pluripotent stem cells) used are negative for surface antigens such as CD34 and CD45 and positive for surface antigens such as CD29 and CD90, and can be simultaneously differentiated into human hepatocytes and immune cells. A liver and immune cell dual-humanized murine model can be constructed simply by transplanting a single type of stem cell. However, the existing humanized models were developed through the co-transplantation of two kinds of cell (human fetal hepatocytes and syngeneic CD34⁺ haematopoietic stem cells (HSCs) or miss-matched human adult hepatocytes and HSCs). When the two kinds of cells are simultaneously transplanted to the mice, the order of transplantation of the two types of cell and the mutual repulsion of the different origins cells need to be considered. If the transplantation sequence, time interval and cell numbers ratio of the two types of cell are improper, it would easily result in low transplantation efficiency, i.e., the two types of cell are difficult to colonize in the mice at the same time. Meanwhile, the present invention develop a novel dual humanized murine model with efficient chimerism of human liver cells and human immune cell lineages only using a single transplantation of stem cell, and can overcome the limitation existing in the current humanized murine model. Moreover, a single type of stem cell transplantation has a higher efficiency, and there are no rejections of the hepatocytes and immune cells simultaneously differentiated in the murine. The dual-humanized murine model obtained by a single type of stem cell transplantation is more stable. This is beneficial to the study of the pathogenesis of natural human HBV infection, as well as of the development, efficacy and drug resistance of new antiviral drugs. This is not achievable by the existing humanized murine model, and cannot be derived by one skilled in the art based on existing knowledge.

In terms of the selection of the strains of the experimental murine, the existing technique for constructing the humanized murine model only uses immunodeficient mice, and is not applicable for normal immune mice. Meanwhile, the strains of experimental murine used in the present invention are in a broad range, including normal immune mice, immunodeficient mice, immune normal rat, or immunodeficient rat. Since the transplanted cells (such as CD34⁺ cells) used in the existing technique for constructing the humanized murine model are not immunological tolerant, mice with normal immune system will immunologically reject CD34⁺ cells. That is, the immune systems of the mice clear these transplanted cells. Therefore, the existing humanized murine model can only select mice with deficient immune system and cannot select normal immune mice. Meanwhile, the human stem cells used in the present invention have immunological tolerance and no rejection reaction. After transplanting these cells into normal murine, they coexist with the murine immune system and are not cleared, so that it can select normal immune murine which are more readily available.

The existing humanized murine model is only permissive for studying HBV or HCV infection and generating a mild immune response against the virus, but complete HBV or HCV disease progression to end-stage liver diseases (such as liver cirrhosis and hepatocellular carcinoma) has not been observed. Further translation is also critically limited by ethical issues and a shortage of available fetal donor hepatocytes with syngeneic HSCs. Meanwhile, for the first time, the present invention delineate the natural disease progression of human HBV infection, the disease progression of hepatitis B cirrhosis and the occurrence and development of hepatitis B related hepatocellular carcinoma. Following HBV infection, the present invention provides a new platform for investigating the full viral life cycle, including the production of HBV DNA, covalently closed circular DNA (cccDNA), surface antigen (HBsAg), e antigen (HBeAg), core antigen (HBcAg) and HBV-induced human immune and inflammatory responses. This series of studies has helped to solve the world's unsolved problems such as hepatitis B, liver cirrhosis and hepatocellular carcinoma. Based on existing knowledge (or common knowledge), one skilled in the art is unable to deduce the animal model of the present invention that can simulates the natural infection of human HBV, the pathogenesis of hepatitis B cirrhosis and the hepatitis B related hepatocellular carcinoma.

In order to elucidate the biological characteristics of hepatitis B virus and the specific mechanism of morbidity of chronic hepatitis B, the present invention has studied the biochemical indicators, immunohistochemistry, gene expression levels, proteomics, and various other aspects. It was found that by inducing liver damage, transplanting human bone marrow mesenchymal stem cells, and injecting hepatitis B viruses, a humanized murine model of chronic hepatitis B may be established. The unique advantage of this model is that such a model murine has a human-derived immune system differentiated from stem cells, and human-derived hepatocytes which form chimera in the murine liver. Human-derived lymphocytes expressing early markers and human-derived hepatocytes expressing hepatocytic surface markers are present in vivo in the murine model. After the injection of hepatitis B viruses, integration of the hepatitis B viral DNA may be found in the genome of the chimeric human-derived hepatocytes in the murine liver. Persistent and stable hepatitis B viruses may be found in the murine serum. The viruses isolated therefrom may continue to infect healthy humanized murines. This shows that this type of viral particle is intact and infectious. After the completion of the hepatitis B viral infection, a continuous increase in transaminase may be observed at the biochemical level, indicating the presence of a persistent chronic liver inflammation. This model may be used for studying the entire natural history of disease of chronic hepatitis B, and the humanized immune response during the process of the chronic infection of hepatitis B virus. Comparing to the existing animal models, the humanized murine model of chronic hepatitis B has apparent advantages. Comparing to the primate model, the murine model has shortened the experimental period and simplified the experimental operation. At the same time, the murine model has greatly reduced the experimental costs. Comparing to the duck model of chronic hepatitis B, murine being mammal is more closely related to human The murine model may be used for studying human hepatitis B virus directly. Comparing to the other transgenic mouse models of hepatitis B viral infection, human-derived hepatocytes and a human-derived immune system based on stem cell transdifferentiation are established in vivo in this murine model. The human-derived immune system may identify and attack the HBV-infected human-derived hepatocytes, thereby causing inflammatory damage to the liver. This model simulates, to the greatest extent, the interaction between hepatitis B virus in the human body and the human immune system, and provide a unique platform for studying the immunopathogenesis of HBV infection and developing immune therapies for viral clearance.

In addition to the study of the mechanism of hepatitis B viral infection, the humanized murine model of chronic hepatitis B may also be used for research in treatment. In view of the current development in hepatitis B drugs, most of the hepatitis B drugs are based on the cellular level of the liver and lack an effective animal model for a comprehensive evaluation of the drugs. The murine model of the present application is simple and readily available, and is a very good simulation of the response of the human body to hepatitis B viral infection. It is of great significance to the research and development of drugs for treating chronic hepatitis B.

When observed under typical feeding, hepatitis B liver fibrosis, hepatitis B liver cirrhosis and hepatitis B primary liver tumors may occur in this murine model of chronic hepatitis B.

### BRIEF DESCRIPTION OF THE DRAWINGS

FIG. 1 is a graph for the research and development of new antiviral drugs using a humanized mouse model of chronic hepatitis B.

FIG. 2 is a graph for the study of drug resistance to the antiviral drugs using a humanized rat model of chronic hepatitis B.

FIG. 3 is a schematic diagram of the study of the pathogenesis of chronic hepatitis B using a humanized rat model of chronic hepatitis B.

FIG. 4 is a graph for the research and development of new anti-fibrosis drugs using a humanized rat model of liver fibrosis and liver cirrhosis.

FIG. 5 is a schematic diagram of the study of the pathogenesis of chronic hepatitis B primary liver tumors using a humanized mouse model of hepatitis B primary liver tumors.

### DETAILED DESCRIPTION OF THE EMBODIMENTS

The present invention discloses a humanized murine model of chronic hepatitis B constructed based on stem cell technology. The technical solution of the present invention is further explained below:

A humanized murine model of chronic hepatitis B constructed using stem cells, wherein the murine model is obtained through the following steps:
1) obtaining human stem cells, wherein the origin of the human stem cells are stem cells isolated and cultured from healthy human, or commercialized cell line;
2) transplanting the human stem cells into a murine with liver damage such that 1×10⁴⁻⁸ of the transplanted human stem cells are present in the murine in vivo; wherein the murine liver damage includes chemical liver damage caused by chemical drugs and physical liver damage caused by surgery; wherein the murine is a normal mouse, an immunodeficient mouse, a normal rat, or an immunodeficient rat;
3) chimera of human-derived hepatocytes: the transplanted human stem cells differentiate into human-derived hepatocytes in the murine liver, wherein the stem cells may express human hapatocytic markers being HSA, HLA, ALB, NTCP, or the like;
4) humanized immune system: the transplanted human stem cells differentiate and form a human-derived immune system in the murine in vivo (liver, blood, spleen, bone marrow, etc.), wherein the human-derived immune system differentiated and formed from the transplanted stem cells may detect immune cells expressing human lymphocytic markers including CD45⁺/CD4⁺/CD8⁺/CD3⁺/CD19⁺/CD20⁺/CD68⁺/NKp46⁺ and the like;
5) infecting the humanized murine with HBV;
6) confirming the HBV infection by analyzing HBV DNA, HBsAg, HBeAg, HBcrAg

HBsAb, HBeAb and HBcAb in serology, HBV cccDNA in hepatocytes and detecting intact HBV particles in persistent and stable presence in the murine in vivo; and
7) confirming the liver disease: hepatitis is confirmed when symptoms such as punctate necrosis are present in the liver of the murine model; liver fibrosis is confirmed when inflammation and fibrosis are present in the liver of the murine model; liver cirrhosis is confirmed when characteristics of liver cirrhosis such as change in pseudo lobule are found; liver tumors is confirmed when lesions of primary liver tumors observable by histology and imaging are present in the murine model in vivo.

The humanized murine model of chronic hepatitis B constructed using stem cells according to the present invention may be used for the research and development of new antiviral drugs, the study of drug resistance to the antiviral drugs, and the study of the pathogenesis of chronic hepatitis B.

The specific steps for preparing the murine model are as follows:

### I. Obtaining Human Stem Cells

1. Isolating and culturing human stem cells
   1) Obtaining purified human stem cells.
   2) Culturing and subculturing the stem cells.
   3) Incubating in an incubator at 20° C. to 40° C., 2% to 10% CO₂.
2. Obtaining commercialized isolated or frozen human stem cells or cell lines.
   II. Transplanting stem cells into murines with liver damage
1. Obtaining different strains of test murine. Examples of test murine include normal mouse, immunodeficient mouse, normal rat, and immunodeficient rat.
2. Establishing a murine model of liver damage by administering liver-damaging drugs through intraperitoneal injection, intramuscular injection, peripheral intravenous injection, oral administration, or gastric administration, or by partial hepatic resection by surgery.
3. Transplanting 1×10⁴⁻⁸ stem cells by means of peripheral intravenous injection, portal vein injection, spleen injection, or liver injection.
   III. HBV infected humanized murines
1. Injecting each murine with hepatitis B virus through peripheral intravenous injection, subcutaneous injection, intramuscular injection, or intraperitoneal injection.
2. Detecting the viral load once within 3-30 days after infection of the murines and confirming that the model is established successfully; or detecting the viral load once within 3-30 days after infection of the murines, and testing the viral load in several times to confirm that the model is established successfully.

### Definitions

The pathogenesis of the related hepatitis, liver fibrosis and the like caused by HBV, and the HBV virus clearance are all inflammatory responses mediated by the host immune response. Specific cytotoxic T lymphocytes (CTL), macrophages, dendritic cells and natural killer cells induce liver inflammation and fibrosis. In particular, specific cytotoxic CD8⁺ T cells play an important role in the pathogenesis of liver inflammation and viral clearance. Interferon (IFN)-γ is a product of activated CD8⁺ T cells that can induce nitric oxide production to prevent the formation of HBV RNA capsids with replicating ability in hepatocytes in a kinase and proteasome dependent manner This process plays a major role in virus clearance. At the same time, virus-specific CD8⁺ T cells migrate to the liver parenchyma and recruit non-antigen-specific polymorphonuclear and mononuclear inflammatory cells, causing hepatocyte apoptosis. A dysfunctional CD8⁺ T cell response does not result in IFN-γ secretion to eliminate HBV and can induce persistently mild damage to hepatocytes and non-parenchymal cell proliferation, leading to chronic HBV infection. Hepatitis B-associated liver fibrosis is a repairing response to inflammatory injury of the liver under HBV stimulation. The activation of liver stellate cells leads to the accumulation of inflammatory cells and the secretion of various cytokines, which eventually leads to excessive deposition of extracellular matrix, and in turn forms liver fibrosis or cirrhosis. However, the specific pathogenesis of chronic hepatitis B, hepatitis B-associated liver fibrosis and liver tumors is still unclear. Accurate research can be performed using the humanized chronic hepatitis B murine model disclosed in the present invention.

Hepatitis B surface antigen (HBsAg) clearance and hepatitis B secretory antigen (HBeAg) seroconversion are considered to be markers of effective treatment for hepatitis B. The drugs currently approved for the treatment of chronic hepatitis B virus are divided into two categories. The first category is the immunomodulator, including interferon α (IFN-α) and pegylated interferon α. The other category is the nucleoside analog and nucleotide analog prodrug, nucleoside analog including lamivudine, telbivudine and entecavir, nucleotide analog prodrug including adefovir dipivoxil and tenofovir fumarate. However, the ultimate goal in the treatment of hepatitis B is to eliminate nuclear cccDNA or to inhibit the transcriptional activity of cccDNA. The drugs currently used still cannot clear the cccDNA that is latent in the liver. The present invention discloses a humanized chronic hepatitis B murine model constructed based on one type of stem cell technique, which provides a safe and stable preclinical research site for drugs eliminating nuclear cccDNA.

Currently, the treatment route of liver fibrosis is by inhibiting the inflammatory response of the liver, such as the use of hormones, to block the expression of cytokines; by regulating the TGF-β signaling pathway, inhibiting the expression of certain genes, and inhibiting the activation of liver stellate cells, thereby inhibiting the synthesis of the eventual collagen; and by increasing the expression of the metalloproteinase and promoting the degradation of the deposited matrix. However, the commonly used animal models of liver cirrhosis at the present time, such as surgery (such as common bile duct ligation) and chemical drugs (carbon tetrachloride, dimethyl nitrosamine, etc.), are caused by non-hepatitis B virus. Therefore, the mechanism and outcome of the disease are very different from the liver cirrhosis caused by hepatitis B, and they are not suitable for the study of the mechanism and treatment of hepatitis B liver fibrosis and cirrhosis. So far, there are still no drugs with well-defined mechanism for treating this type of disease.

At present, due to the fact that hepatocellular carcinoma models are mostly caused by chemical drugs or transfection of oncogenes, there are few studies on the mechanism of occurrence, development and treatment of liver tumors caused by hepatitis B. Based on the present model, the occurrence and development of hepatitis B primary liver tumors can be simulated. Through the multi-omics association analysis of proteomics and genomics, the changes in genomics and proteomics in the process of liver tumors metastasis can be found. Identification of genes and proteins with significant differences and analysis of related pathways can reveal the mechanism of liver tumors metastasis. The use of humanized hepatitis B primary liver tumors mouse model to study the mechanism of liver tumors is expected to provide new ideas for the prevention and treatment of liver tumors.

### EXAMPLES

The technical solution of the present invention will be further described below in accordance with the drawings and specific embodiments.

### Example 1

The Research and Development of New Antiviral Drugs Using a Humanized Mouse Model of Chronic Hepatitis B
1. 150 humanized mice with chronic hepatitis B were randomly divided into three groups with 50 mice in each group. Prior to treatment, the viral load of hepatitis B viruses was about 1×10⁷ and there was no significant difference between the three groups.
2. Therapeutic dose of a new antiviral drug was administered to the test group. Tenofovir (100 mg/kg), a first-line nucleoside analogue antiviral drug, was orally administered to the control group 1. Antiviral treatment by interferon injection was administered to the control group 2.
3. At 1 week, 2 weeks, 4 weeks, 8 weeks, 12 weeks and 24 weeks after the treatment, the viral load of HBV in each group of mice was detected, respectively. Adverse effects (such as renal function, myocardial enzymes, etc.) in other major organs were also detected.
4. The curve of the change in virus load in the test group of mice (i.e., new antiviral drug) was compared with those of the control group 1 (i.e., tenofovir) and the control group 2 (i.e., interferon), thereby obtaining the difference in antiviral effect between the new antiviral drug, the existing first-line antiviral drug tenofovir, and interferon.
5. The adverse effects of the new antiviral drug were drawn through the observation of side effects, which lays the foundation for conducting a phase 1 clinical trial.

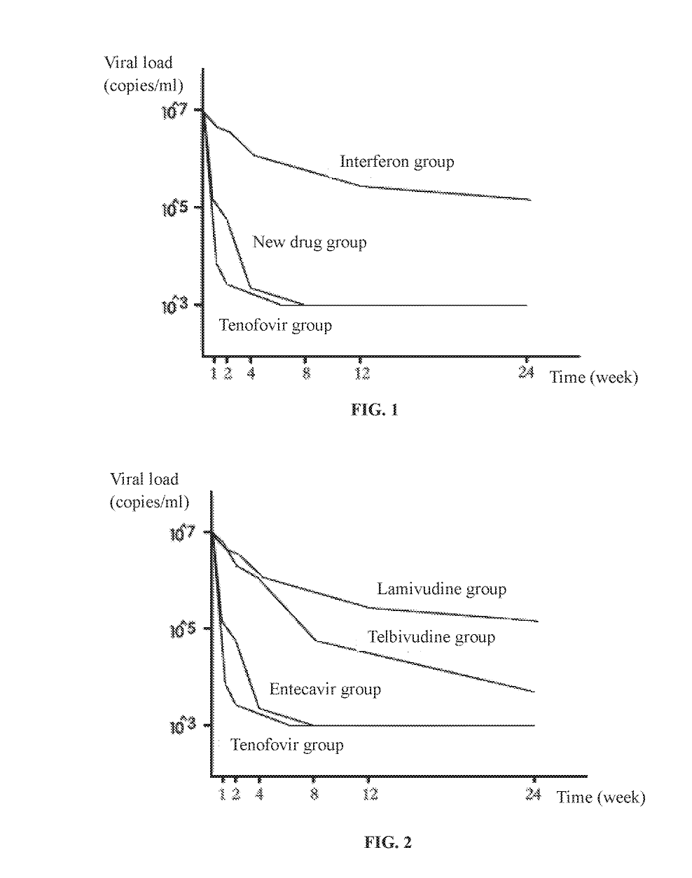

FIG. 1 is a graph for the development of new antiviral drugs using the humanized mouse model of chronic hepatitis B. It is demonstrated that the humanized mouse model of chronic hepatitis B may be used for the effective screening and testing of new drugs. In the graph, the drop of the viral titer curve of the new drug group is similar to that of the first-line drug tenofovir group, and is superior to the interferon group.

### Example 2

The Study of Efficacy of and Drug Resistance to the Antiviral Drugs Using a Humanized Rat Model of Chronic Hepatitis B
1. 200 humanized rats with chronic hepatitis B were randomly divided into four groups. There were no significant differences in age, gender, weight and baseline of HBV viral load among the groups.
2. Therapeutic dose of commonly used antiviral drugs lamivudine, telbivudine, entecavir and tenofovir were administered to each of the four groups, respectively.
3. At 1 week, 2 weeks, 4 weeks, 8 weeks, 12 weeks and 24 weeks after the treatment, the viral load of HBV in each group of rats was detected, respectively.
4. Curves were plotted based on the viral load in each group at each time point. The difference between the groups was compared to evaluate the efficacy of and drug resistance to existing commonly used antiviral drugs.

FIG. 2 is a graph of the viral load in each group for the study of drug resistance to the antiviral drugs using the humanized rat model of chronic hepatitis B. It is demonstrated that the humanized rat model of chronic hepatitis B may be effectively used for the study of drug resistance to the antiviral drugs. In the graph, the drop of the viral titer curves of the tenofovir group and the entecavir group are superior to those of the telbivudine group and the lamivudine group.

### Example 3

The Study of the Pathogenesis of Chronic Hepatitis B Using a Humanized Rat Model of Chronic Hepatitis B
1. 50 humanized rats with chronic hepatitis B were prepared. The time at which the viruses were injected was defined as the starting point.
2. On day 1, day 3, day 5, day 7, day 14, day 30, day 60, day 90, day 180 and day 360 from the starting point, 5 rats were sacrificed, respectively. Samples such as serum and liver tissue were collected.
3. Spectrum changes in the genome and proteome developed due to hepatitis B infection and chronic hepatitis B were identified through multi-omic association analysis such as proteomics and genomics.
4. The genes and proteins showing significant differences were identified and the relevant pathways were analyzed to reveal the pathogenesis of chronic hepatitis B.

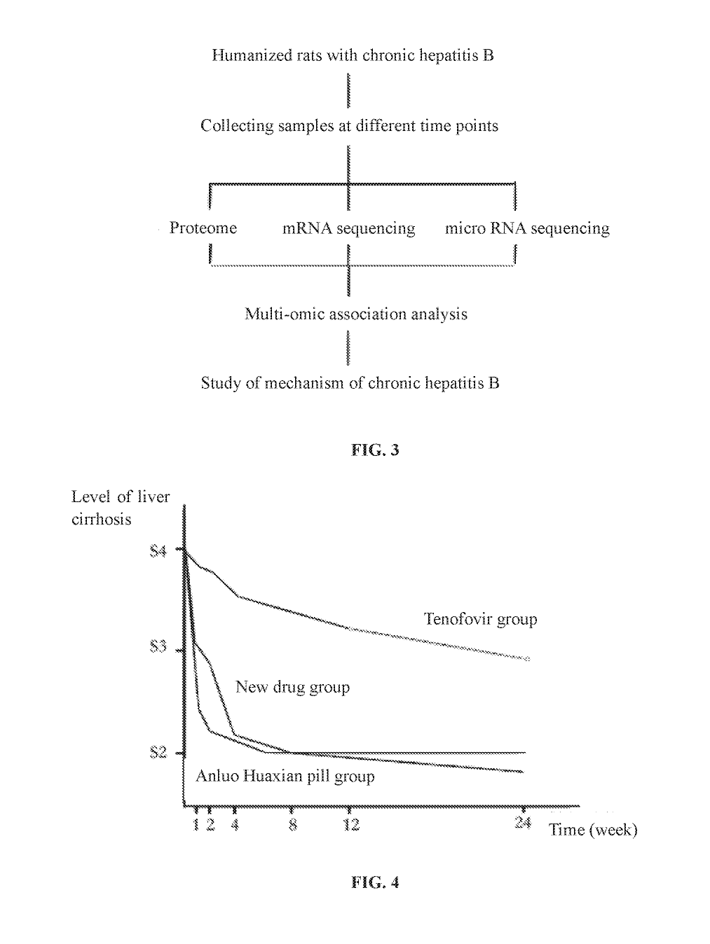

FIG. 3 is a schematic diagram of the study of the pathogenesis of chronic hepatitis B using the humanized rat model of chronic hepatitis B. From the perspective of proteomics and genomics, such a model may study the mechanism of chronic hepatitis B through analysis of multi-omic association.

### Example 4

The Research and Development of New Anti-Fibrosis Drugs Using a Humanized Rat Model Of Liver Fibrosis and Liver Cirrhosis
1. When it was clear that inflammation and fibrosis present in the liver of the humanized chronic hepatitis B rat model was liver fibrosis, and when there was characteristic change of pseudo lobule in cirrhosis, this model progressed to the rat model of humanized liver fibrosis and cirrhosis. 75 humanized rats with liver fibrosis and liver cirrhosis were randomly divided into three groups with 25 rats in each group. Prior to treatment, there was no significant difference in the level of liver fibrosis among the three groups.
2. Therapeutic dose of a new anti-fibrosis drug was administered to the test group. Anluo Huaxian pill and the first-line antiviral drug tenofovir were orally administered to each of the control groups, respectively.
3. At 1 week, 2 weeks, 4 weeks, 8 weeks, 12 weeks and 24 weeks after the treatment, the level of liver fibrosis in the rats was measured, respectively. Adverse effects (such as renal function, myocardial enzymes, etc.) in other major organs were also detected.
4. The level of liver fibrosis in the test group of rats (i.e., new antiviral drug) was compared with those in the control groups (i.e., Anluo Huaxian pill and tenofovir), thereby obtaining the difference in anti-fibrosis effect between the new anti-fibrosis drug, Anluo Huaxian pill, and the existing first-line antiviral drug tenofovir.
5. The adverse effects of the new anti-fibrosis drug were drawn through the observation of side effects, which lays the foundation for conducting a phase 1 clinical trial.

FIG. 4 demonstrates that the humanized rat model of chronic hepatitis B liver cirrhosis may be used for the effective screening and testing of new drugs. In the graph, the drop of the curve of the level of liver cirrhosis in the new drug group is similar to but slightly better than that in the Anluo Huaxian pill group, and is superior to the first-line drug tenofovir group.

### Example 5

The Study of the Mechanism of Liver Tumors Using a Humanized Mouse Model of Hepatitis B Primary Liver Tumors
1. When histological and imaging observable primary liver tumors lesions were present in the mouse model of humanized chronic hepatitis B, this model progressed to the mouse model of humanized hepatitis B primary liver tumors. 100 humanized mice with hepatitis B primary liver tumors were fed with conventional feeding with no treatment intervention.
2. The humanized hepatitis B primary liver tumors model was grouped according to the progress of the tumor metastasis. Mice with single liver tumors lesion only and no metastasis were group into the first group; mice with multiple liver tumors lesions and no metastasis were grouped into the second group; and mice with liver tumors lesions and other organ metastasis were grouped into the third group. There were approximately 25 mice in each group. Samples such as serum, various organ tissues and lymph nodes were collected.
3. Spectrum changes in the genome and proteome during liver tumors metastasis were identified through multi-omic association analysis such as proteomics and genomics.
4. The genes and proteins showing significant differences were identified and the relevant pathways were analyzed to reveal the mechanism of liver tumors metastasis.

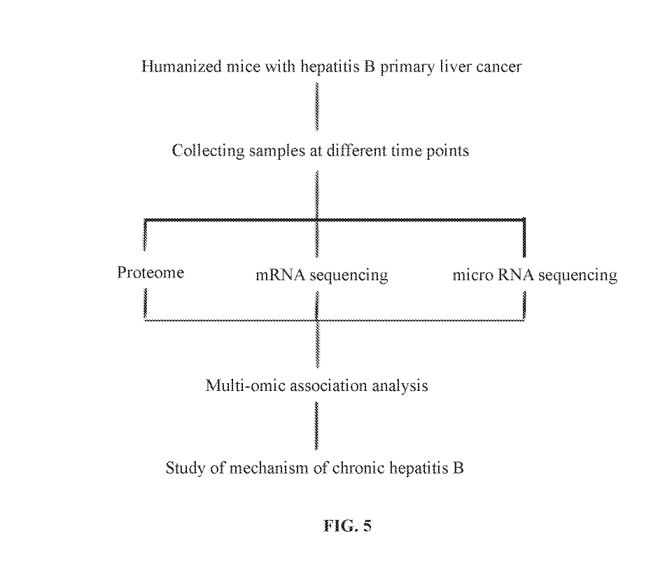

As shown in FIG. 5, from the perspective of proteomics and genomics, such a mouse model of liver tumors may be used for studying the mechanism of hepatitis B primary liver tumors through analysis of multi-omic association.

The above examples are merely the preferred embodiments of the present invention, and the present invention is not limited to the above embodiments. Other improvements and changes directly deduced or conceived of by one skilled in the art without departing from the gist and concept of the present invention should also be considered as being included in the scope protected by the present invention.

What is claimed is:

1. A humanized murine model of chronic hepatitis B constructed using human stem cells, wherein the humanized murine model is obtained by the following steps:
(a) obtaining the human stem cells;
(b) transplanting the human stem cells into a murine with liver damage to achieve presence of 1×10⁴⁻⁸ of the human stem cells in the murine in vivo;
(c) forming chimera of human-derived hepatocytes, wherein the human stem cells transplanted in the step (b) differentiate into the human-derived hepatocytes in a liver of the murine; and forming a humanized immune system, wherein the human stem cells transplanted in the step (b) differentiate and form the human-derived immune system in the murine in vivo;
(d) infecting the murine with hepatitis B virus (HBV); and
(e) confirming a HBV infection and a liver disease in the murine.

2. The humanized murine model according to claim 1, wherein the human stem cells originated from stem cells isolated and cultured from a healthy human, or a commercialized cell line.

3. The humanized murine model according to claim 1, wherein the liver damage comprises chemical liver damage caused by a chemical drug, physical liver damage caused by surgery or both; wherein the murine is a normal mouse, an immunodeficient mouse, a normal rat, or an immunodeficient rat.

4. The humanized murine model according to claim 1, wherein in the step (c), the human stem cells which differentiate into the human-derived hepatocytes express a human hapatocytic marker selected from the group consisting of HSA, HLA, ALB and NTCP.

5. The humanized murine model according to claim 1, wherein in the step (c), the human-derived immune system formed by the human by the human stem cells is capable of detecting an immune cell expressing a human lymphocytic marker selected from the group consisting of CD45⁺, CD4⁺, CD8⁺, CD3⁺, CD19⁺, CD20⁺, CD68⁺and NKp46⁺.

6. The humanized murine model according to claim 1, wherein in the step (e), the HBV infection is confirmed by detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

7. The humanized murine model according to claim 2, wherein in the step (e), the HBV infection is confirmed by detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

8. The humanized murine model according to claim 3, wherein in the step (e), the HBV infection is confirmed by detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

9. The humanized murine model according to claim 4, wherein in the step (e), the HBV infection is confirmed by detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

10. The humanized murine model according to claim 5, wherein in the step (e), the HBV infection is confirmed by detecting intact HBV particles in persistent and stable presence in the murine in vivo; wherein the liver disease is selected from the group consisting of hepatitis, liver fibrosis developed from the hepatitis, liver cirrhosis developed from the hepatitis, and liver tumors developed from the hepatitis.

11. The humanized murine model according to claim 6, wherein the hepatitis is confirmed when a symptom comprising punctate necrosis is present in the liver of the murine; the liver fibrosis is confirmed when inflammation and fibrosis are present in the liver of the murine; the liver cirrhosis is confirmed when a characteristic of the liver cirrhosis comprising change in pseudo lobule is found; the liver tumors is confirmed when a lesion of a primary liver tumors observable by histology and imaging is present in the murine in vivo.

12. The humanized murine model according to claim 7, wherein the hepatitis is confirmed when a symptom comprising punctate necrosis is present in the liver of the murine; the liver fibrosis is confirmed when inflammation and fibrosis are present in the liver of the murine; the liver cirrhosis is confirmed when a characteristic of the liver cirrhosis comprising change in pseudo lobule is found; the liver tumors is confirmed when a lesion of a primary liver tumors observable by histology and imaging is present in the murine in vivo.

13. The humanized murine model according to claim 8, wherein the hepatitis is confirmed when a symptom comprising punctate necrosis is present in the liver of the murine; the liver fibrosis is confirmed when inflammation and fibrosis are present in the liver of the murine; the liver cirrhosis is confirmed when a characteristic of the liver cirrhosis comprising change in pseudo lobule is found; the liver tumors is confirmed when a lesion of a primary liver tumors observable by histology and imaging is present in the murine in vivo.

14. The humanized murine model according to claim 9, wherein the hepatitis is confirmed when a symptom comprising punctate necrosis is present in the liver of the murine; the liver fibrosis is confirmed when inflammation and fibrosis are present in the liver of the murine; the liver cirrhosis is confirmed when a characteristic of the liver cirrhosis comprising change in pseudo lobule is found, normal central-portal relationships is lossed; the hepatocellular tumors is confirmed when a lesion of primary liver tumors observable by tissue biopsy and imaging is present in the murine in vivo.

15. The humanized murine model according to claim 10, wherein the hepatitis is confirmed when a symptom comprising punctate necrosis is present in the liver of the murine; the liver fibrosis is confirmed when inflammation and fibrosis are present in the liver of the murine; the liver cirrhosis is confirmed when a characteristic of the liver cirrhosis comprising change in pseudo lobule is found; the liver tumors is confirmed when a lesion of primary liver tumors observable by histology and imaging is present in the murine in vivo.

16. A method of researching and developing an antiviral drug, an anti-fibrosis drug, or an anti-tumor drug, comprising:
screening and testing the antiviral drug, anti-fibrosis drug, or anti-tumor drug using the humanized murine model according to claim 1.

17. A method of studying drug resistance to an antiviral drug, comprising:
testing the drug resistance to the antiviral drug using the humanized murine model according to claim 1.

18. A method of studying pathogenesis of chronic hepatitis B, comprising:
analyzing the pathogenesis of the chronic hepatitis B using the humanized murine model according to claim 1.
